(19) United States
(12) Patent Application Publication
(10) Pub. No.: US 2006/0257342 A1
(43) Pub. Date: Nov. 16, 2006

(54) Long-wearing glossy cosmetic composition

(76) Inventors: Weilin Mu, Albertson, NY (US); Dexin Luo, Fresh Meadows, NY (US); Linda McKenna, North Babylon, NY (US); Shahan Nazar, Garden City, NY (US); George J. Stepniewski, Melville, NY (US); Tian Xiang Wang, Dix Hills, NY (US)

Correspondence Address: THE ESTEE LAUDER COS, INC, 125 PINELAWN ROAD, MELVILLE, NY, 11747, US

(21) Appl. No.: 11/127,938

(22) Filed: May 12, 2005

(51) Int. Cl. A61K 8/81

(52) U.S. Cl. 424/63; 424/70.16; 424/64

(57) ABSTRACT

A transfer-resistant, single phase aqueous cosmetic composition is provided. The composition contains a water-soluble, film-forming acrylic acid- or methacrylic acid-containing acrylates copolymer and a water-soluble ester. The composition is water- and oil-resistant upon drying on the skin, and exhibits high gloss and long-wearing and transfer-resistant properties. Compositions containing pigment also demonstrate high color intensity. The compositions are useful as eyeliner, mascara, lipgloss, lipliner, and other cosmetic products.

### BACKGROUND OF THE INVENTION

1. Field of the Invention

The present invention relates to cosmetic compositions for application to the skin, including the lips and the eyelids, and to the eyelashes. The present invention also relates to cosmetic compositions, which, when dried, after application to the skin or eyelashes, exhibit excellent water- and oil-resistance and long-wear properties, and which do not readily transfer to clothing or to other surfaces. The invention also relates to cosmetic compositions that contain substantially no oils, and yet demonstrate high gloss and color intensity. The invention further relates to aqueous cosmetic compositions that do not require emulsifiers or surfactants which are typically needed to stabilize emulsion formulations.

2. Background of the Invention

Vivid color, shine and long wear are desirable properties for makeup products, particularly for eyeliner and lipgloss.

Eyeliner is a particularly desirable beautifying product. It is preferred that eyeliner be intensely colored and shiny. To achieve an intensely colored black eyeliner, for example, many prior art compositions use carbon black. However, the use of carbon black results in a cosmetic product with a less than desirable level of gloss. The use of black iron oxide in other typical eyeliner formulations containing acrylates polymers and/or copolymers has resulted in products that lacked a desirable level of color intensity, since the polymers, in most cases, are white and opaque. When the polymers dry, the film is matte or exhibits a metallic or plastic shine, which generally is not desired by consumers.

However, by using water-soluble plasticizers in combination with water-soluble polymers, a transparent matrix or base is obtained that, after drying, forms a film with an oil-like shine. The high gloss intensifies the hue of colorant-containing compositions, providing a rich, deep color.

A long-wearing product that resists smudging, running and/or or fading upon exposure to oils and perspiration or tears also would be appreciated by consumers. The present invention provides transfer-resistant and water- and oil-resistant film that is long-wearing.

Non-transfer acrylates polymer-containing cosmetic compositions typically have been provided in the form of emulsions containing oils, surfactants, and/or emulsifiers or anhydrous formulations containing volatile components such as oils or alcohols. Nevertheless, products that do not contain emulsifiers, surfactants, oils and alcohols which may irritate or dry the skin, including the lips, of the user have become more desirable.

Moreover, surfactants and/or wetting agents typically have been used in cosmetic compositions for dispersing solid pigments. Such agents tend to absorb moisture because of their chemical natures. Thus the integrity of the film formed after the composition has been applied and has dried may be compromised. In the compositions of the subject invention, however, the pigments, particularly metallic oxides, are readily dispersible in the aqueous system without dispersing agents or surfactants due to the unique combination of water-soluble plasticizers and acrylates copolymers. The pigments interacting with the water-soluble components form ultra-fine particles which increase the smoothness of the film formed. An eyeliner with as high coverage as a composition using carbon black is provided, but with a dewy appearance which is much preferred by consumers. The water- and oil-resistance properties of the compositions enhance their transfer-resistance.

The lipliners and lipglosses prepared according to the invention are shiny and brilliantly colored. Moreover, the water- and oil-resistance and transfer-resistance properties of the lipliner products produced enable the user to enlarge or reduce the appearance of the size of the lips which is also highly desired by consumers.

### SUMMARY OF THE INVENTION

The invention relates to cosmetic compositions containing acrylates copolymers which are truly water-soluble yet surprisingly demonstrate water-resistance and transfer-resistance upon drying on the skin, including the lips and the eyelids, and on the eyelashes. The compositions of the invention contain water-soluble film forming acrylic acid- or methacrylic acid-containing acrylates copolymer and a water-soluble plasticizer in an aqueous medium. The invention compositions contain substantially no oils and yet exhibit high gloss and color intensity. The aqueous compositions of the invention do not require surfactants and emulsifiers which are used to maintain the stability of two-phase formulations. The compositions of the invention also are water- and/or oil-resistant.

The invention also is concerned with a method of improving the transfer-resistance, the shine and/or the color intensity of a cosmetic composition by combining a water-soluble film-forming acrylic acid- or methacrylic acid-containing acrylates copolymer and a water-soluble plasticizer in an aqueous medium in the substantial absence of oils.

The invention also concerns a method for redefining the lipline which comprises providing a liplining composition comprising a transfer-resistant single phase aqueous composition comprising a water-soluble film forming acrylic acid- or methacrylic acid-containing acrylates copolymer and a water-soluble plasticizer and a pigment, preferably wherein the composition is a natural lip color composition, and tracing the composition, for example with a liplining brush, just outside or just inside the lipline, allowing the product to dry, and applying within the traced line a desired shade. Optionally, a lipgloss, comprising a transfer-resistant single phase aqueous composition comprising a water-soluble film forming acrylic acid- or methacrylic acid-containing acrylates copolymer, a water-soluble plasticizer and optionally a pigment, may be applied over the desired shade.

### DETAILED DESCRIPTION OF THE INVENTION

The novel cosmetic compositions of the invention are single phase aqueous formulations containing a water-soluble film-forming acrylates copolymer, a water-soluble plasticizer, and optionally a pigment. By “single phase” it is intended that the composition is in a stable homogeneous form rather than in the form of a heterogeneous water-in-oil or oil-in-water emulsion. The film-forming acrylates copolymer and plasticizer are solublized in that single phase. Pigments, if any are present, are dispersed throughout the liquid in sufficiently small particles to remain stable in the composition. The copolymer, once dried, is neither water- nor oil-soluble. The unique combination of a film-forming water-soluble acrylates copolymer and a water-soluble plasticizer in an aqueous medium provides a product which, upon drying on the skin, including the lips, demonstrates high color intensity, high gloss and excellent long-wearing, transfer-resistant properties.

The compositions of the invention contain substantially no (less than 0.2 weight percent, if present) hydrophobic oils, and do not require the surfactants and emulsifiers which are employed to stabilize two-phase (emulsion) compositions. Typical hydrophobic oils include those disclosed, for example, in U.S. Pat. Nos. 5,843,407 and 6,780,422, the entire disclosures of which are incorporated by reference, and in the International Cosmetic Ingredient Dictionary and Handbook, twelfth edition, 2004, which also is hereby incorporated by reference. Oils are those materials which are organic substances that are liquid at ambient temperature. They are esters, triglycerides, hydrocarbons and silicones.

Emulsifiers and surfactants are typically required for use in cosmetic emulsions, which contain normally immiscible water and oil phases, to disperse one of the water and the oil phases in the other phase. Typical emulsifiers and surfactants are disclosed in McCutcheon's, Vol. 1: Emulsifiers and Detergents, North American Edition, 2004, the entire disclosure of which is hereby incorporated by reference. When emulsifiers and/or surfactants are present in cosmetic compositions and applied, for example, to the eyelid, the cosmetic film formed when the composition has dried still can bind water (e.g. perspiration) and oil (skin oils), thereby becoming solubilized, resulting in smudging and reduced length of wear. In contrast to emulsion compositions, the compositions of the invention do not contain hydrophobic oils; and, therefore, require no emulsifiers and surfactants to stabilize the formulations. The single phase, aqueous compositions of the invention contain truly water-soluble, film-forming copolymers which, when applied to the skin of the eyelid, for example, are not easily solublized, after drying, by either water or oil, and therefore are long-wearing, smudge-resistant and transfer-resistant. If present in the aqueous compositions of the invention, any material which demonstrates emulsifier or surfactant properties will have an HLB of less than 12.

Preferably, the water-soluble acrylates copolymer is present in the composition in an amount in the range of about 1 to about 95 percent, more preferably, in an amount of about 5 to about 70 percent, and even more preferably, in an amount of about 10 to about 30 percent, by weight of the total composition.

The water-soluble plasticizer preferably is present in the composition in an amount in the range of about 1 to about 60 percent, and more preferably, in an amount of about 2 to about 20 percent, by weight of the total composition.

The ratio of the acrylates copolymer to the water-soluble plasticizer in the invention compositions is greater than about 1:1, preferably about 2:1 to 20:1, and more preferably about 2:1 to 4:1.

The pigment, if present in the invention composition, is preferably present in an amount in the range of about 1 to about 20 percent, and more preferably is present in the amount of about 5 to about 15 percent, based on the total weight of the composition.

The water-soluble acrylates copolymer is an acrylic or methacrylic acid-containing copolymer in which the comonomer is selected from a wide range of alkyl and alkoxyl acrylates and alkyl and alkoxyl methacrylates. Preferred comonomers include those disclosed in U.S. Pat. No. 5,948,419, the entire disclosure of which is herein incorporated by reference. The comonomers preferably are C1-C8 alkyl acrylates or methacrylates or a C1-C4 alkoxy acrylates or methacrylates, and include, but are not limited to, methyl acrylate, methyl methacrylate, ethyl acrylate, ethyl methacrylate, propyl acrylate, propyl methacrylate, isopropyl acrylate, isopropyl methacrylate, tertiary butyl acrylate, tertiary butyl methacrylate, isobutyl acrylate, isobutyl methacrylate, pentyl acrylate, pentyl methacrylate, isopentyl acrylate, isopentyl methacrylate, neopentyl acrylate, neopentyl methacrylate, hexyl acrylate, hexyl methacrylate, isohexyl acrylate, isohexyl methacrylate, heptyl acrylate, heptyl methacrylate, isoheptyl acrylate, isoheptyl methacrylate, octyl acrylate, octyl methacrylate, isooctyl acrylate, isooctyl methacrylate, 2-ethylhexyl acrylate, 2-ethylhexyl methacrylate, 2-hydroxyethyl (meth)acrylate, polyethylene oxide (meth)acrylates, PEG (meth)acrylates, 3-hydroxypropyl (meth)acrylates, 2-hydroxypropyl (meth)acrylates, polypropylene oxide (meth)acrylates, PPG (meth)acrylates, 3-hydroxybutyl (meth)acrylates, polybutylene oxide (meth)acrylates, or a combination of any two or more of the comonomers. Preferred copolymers may include ethyl acrylate/methyl methacrylate/methacrylic acid; ethyl acrylate/methyl methacrylate/acrylic acid; ethyl acrylate/ethyl methacrylate/methacrylic acid; ethyl acrylate/ethyl methacrylate/acrylic acid; methyl acrylate/methyl methacrylate/methacrylic acid; methyl acrylate/methyl methacrylate/acrylic acid; methyl acrylate/ethyl methacrylate/methacrylic acid; and methyl acrylate/ethyl methacrylate/acrylic acid, particularly ethyl acrylate/methyl methacrylate/methacrylic acid and ethyl acrylate/methyl methacrylate/acrylic acid. Most preferred are Covacryl A15® and Covacryl E14® (LCW, France) which are ethyl acrylate/methylmethacrylate/methacrylic acid copolymers.

The water-soluble plasticizer may be selected from polyether derivatives, polyoxypropylene derivatives, glycol and glycol derivatives and glycerin and glycerin derivatives, and combinations thereof. The glycols may include polyethylene glycol, polypropylene glycol and polybutylene glycol. The water-soluble plasticizer preferably is a polyglyceryl ester or a PEG and/or PPG-modified ester, for example, polyglyceryl-3 laurate, PEG-90 diisostearate, PEG/PPG-8/3 laurate, PEG/PPG-8/3 diisostearate, or triisostearoyl polyglyceryl-3 dimer dilinoleate. Most preferably, the water-soluble ester is Hydramol PGPD®, Hydramol PGPL®, Hydramol PGDS®, Hydramol TGL® or Schercemol PTID® (Industrial West, N.J.). While not wishing to be bound by any theory, it is believed that the plasticizer acts like a wetting agent, which physically bonds to (coats) the pigment particles, improving pigment dispersion and contributing to the shine of the composition film when dried. Although a wetting property may be attributable to the plasticizer useful in the compositions of the invention, it is not intended that such compounds fall with the scope of the typical emulsifiers and surfactants used in conventional emulsion compositions, such emulsifiers and surfactants being substantially absent from the compositions of the invention. In fact, the plasticizers used in the present compositions could not be used in an emulsion composition as the major emulsifying agent.

The invention compositions optionally include a pigment selected from cosmetically acceptable inorganic and organic pigments, such as those disclosed in the International Cosmetic Ingredient Dictionary and Handbook, twelfth edition, 2004, the entire disclosure of which is herein incorporated by reference. The inorganic pigments may include red, black, green and yellow iron oxides, titanium dioxide, bismuth oxychloride and the like. The organic pigments may include D&C and FD&C colors. Pigments suitable for use in the invention compositions include those disclosed in U.S. Pat. No. 6,726,900, the entire disclosure of which is incorporated herein by reference. Particularly preferred organic pigments are red, green, blue, yellow, violet, orange, lakes thereof and mixtures thereof, including FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Particularly preferred lakes are formed by the reaction of the organic pigment with a metallic salt such as aluminum, calcium, zirconium, barium, and the like. Suitable reds include pigments from the monoazo, disazo, fluoran, xanthene, or indigoid families or lakes thereof, such as Red 4, 6, 7, 17, 21, 22, 27, 28, 30, 31, 33, 34, 36, and Red 40. Also suitable are lakes of such red pigments. Typically, the metal salts are aluminum, barium, and the like. The yellow pigment may be a pyrazole, monoazo, fluoran, xanthene, quinoline, or salt thereof. Suitable yellows include Yellow 5, 6, 7, 8, 10, and 11, as well as lakes of such yellow pigments. Suitable violets include those from the anthroquinone family, such as Violet 2 and lakes thereof. Examples of orange pigments are Orange 4, 5, 10, 11, or lakes thereof.

The compositions may include further components, such as one or more water-dispersible plasticizers, for example, water-dispersible esters, such as Covaplast® (LCW, France), antimicrobials, preservatives, stabilizers, suspending agents or thickeners, water-soluble actives and combinations thereof.

Typical preservatives which may be used in the invention compositions include, for example, ethylhexylglycerin and caprylyl glycol/phenoxyethanol/hexylene glycol. Other preservatives suitable for use in the compositions are disclosed in the International Cosmetic Ingredient Dictionary and Handbook, twelfth edition, 2004.

Suitable stabilizers, suspending agents or thickeners for use in the invention compositions include, but are not limited to PVP copolymers, such as ammonium acrylodimethyltaurate/VP polymer; cellulose derivatives, for example, hydroxyethylcellulose and sodium carboxymethylcellulose; acrylates, such as glyceryl polyacrylate; polysaccharide derivatives, for example, sclerotium gel; modified corn starch; silicates, such as magnesium aluminum silicate and sodium magnesium silicate; hectorite and derivatives, such as Bentones; and xanthan gum. Additional stabilizers, suspending agents and thickeners are disclosed in McCutcheon's Volume 2: Functional Materials, North American Edition, 2004, the entire disclosure of which is hereby incorporated by reference.

Water soluble actives which may be used in the compositions of the invention include sunscreens (such as Eusolex 232); and antioxidants, for example ginkgo-biloba, beta carotene, green tea, ascorbic acid and derivatives thereof such as sodium ascorbyl phosphate and magnesium ascorbyl phosphate and carnosic acid (rosemary).

The compositions of the invention may be embodied in, for example, eyeliner, mascara, concealer, lip gloss and lipliner.

The following non-limiting examples further illustrate the embodiments of the invention.

### EXAMPLES

### Example 1

### Eyeliner Formulation

TABLE 1

| | |
|---|---|
| | WEIGHT |
| MATERIAL | PERCENT |

sequence 1

| | |
|---|---|
| purified water | 20.00 |
| magnesium aluminum silicate | 0.50 |
| cellulose gum | 0.20 |
| xanthan gum | 0.20 |
| ethylhexylglycerin | 0.25 |

sequence 2

| | |
|---|---|
| purified water | 6.75 |
| ethylhexylglycerin | 0.25 |
| iron oxides | 13.00 |

sequence 3

| | |
|---|---|
| acrylates copolymer (Covacryl A15) | 9.80 |
| acrylates copolymer (Covacryl E14) | 4.00 |
| caprylylglycol/phenoxyethanol/hexylene glycol | 0.13 |
| ethylhexylglycerin | 0.25 |

sequence 4

| | |
|---|---|
| PEG-90 diisostearate (Hydramol PGDS) | 2.70 |
| PEG/PPG-8/3 laurate (Hydramol PGPL) | 1.28 |
| caprylylglycol/phenoxyethanol/hexylene glycol | 0.15 |
| ethylhexylglycerin | 0.25 |

sequence 5

| | |
|---|---|
| kaolin | 5.00 |

sequence 6

| | |
|---|---|
| purified water | 35.30 |
| TOTAL | 100.00 |

With reference to Table 1, pure water and ethylhexylglycerin are heated to 50° C. Magnesium aluminum silicate, cellulose gum and xanthan gum are sprinkled into the water and ethylhexylglycerin mixture separately and stirred until the mixture is gelled. The temperature of the mixture is reduced to 25° C. forming sequence 1. Sequence 2 materials are milled together and added to the sequence 1 mixture. The materials of sequence 3 are added to the above mixture. The materials of sequences 4, 5 and 6 then are sequentially added to the above mixture. The product prepared is intensely black and shiny and transfer-resistant.

In examples 2 through 5 (Tables 2 through 5), the materials of sequence 1 are combined and mixed until uniform, the materials of sequence 2 are milled together and added to the sequence 1 mixture. The materials of each additional sequence are added to the above mixture and mixed together until uniform. The eyeliner formulations of the invention are given below in Tables 2 through 5.

### Example 2

### Eyeliner Formulation

TABLE 2

| | |
|---|---|
| | WEIGHT |
| MATERIAL | PERCENT |

sequence 1

| | |
|---|---|
| acrylates copolymer (Covacryl A15) | 9.80 |
| acrylates copolymer (Covacryl E14) | 4.00 |
| caprylyl glycol/phenoxyethanol/hexylene glycol | 0.25 |
| ethylhexylglycerin | 0.50 |

sequence 2

| | |
|---|---|
| purified water | 6.75 |
| ethylhexylglycerin | 0.25 |
| iron oxides (cosmetic black) | 13.00 |

sequence 3

| | |
|---|---|
| PEG-90 diisostearate (Hydramol PGDS) | 6.00 |
| PEG/PPG-8/3 laurate (Hydramol PGPL) | 6.00 |
| caprylylglycol/phenoxyehtanol/hexylene glycol | 0.25 |
| ethylhexylglycerin | 0.25 |

sequence 4

| | |
|---|---|
| purified water | 52.95 |
| TOTAL | 100.00 |

### Example 3

### Eyeliner Formulation

TABLE 3

| | |
|---|---|
| | WEIGHT |
| MATERIAL | PERCENT |

sequence 1

| | |
|---|---|
| acrylates copolymer (Covacryl E14) | 15.00 |

sequence 2

| | |
|---|---|
| purified water | 36.50 |
| iron oxides (cosmetic black) | 10.00 |

sequence 3

| | |
|---|---|
| kaolin | 30.00 |

sequence 4

| | |
|---|---|
| PEG-90 diisostearate (Hydramol PGDS) | 0.50 |
| PEG/PPG-8/3 laurate (Hydramol PGPL) | 8.00 |
| TOTAL | 100.00 |

### Example 4

### Eyeliner Formulation

TABLE 4

| | |
|---|---|
| | WEIGHT |
| MATERIAL | PERCENT |

sequence 1

| | |
|---|---|
| acrylates copolymer (Covacryl A15) | 15.00 |
| acetyl tributyl citrate, tr trioctyltrimellitate, | 0.50 |
| triethyl citrate | |

sequence 2

| | |
|---|---|
| iron oxides | 12.50 |
| purified water | 7.50 |

sequence 3

| | |
|---|---|
| PEG-90 diisostearate (Hydramol PGDS) | 2.70 |
| PEG/PPG-8/3 laurate (Hydramol PGPL) | 1.28 |

sequence 4

| | |
|---|---|
| kaolin | 6.00 |

sequence 5

| | |
|---|---|
| purified water | 54.52 |
| TOTAL | 100.00 |

### Example 5

### Eyeliner Formulation

TABLE 5

| | |
|---|---|
| | WEIGHT |
| MATERIAL | PERCENT |

sequence 1

| | |
|---|---|
| acrylates copolymer (Covacryl A15) | 9.80 |
| acrylates copolymer (Covacryl E14) | 4.00 |

sequence 2

| | |
|---|---|
| iron oxides | 12.50 |
| purified water | 7.50 |

sequence 3

| | |
|---|---|
| PEG-90 diisostearate (Hydramol PGDS) | 3.00 |

sequence 4

| | |
|---|---|
| kaolin | 6.00 |

sequence 5

| | |
|---|---|
| purified water | 57.20 |
| TOTAL | 100.00 |

### Example 6

### Lip Gloss Formulation

TABLE 6

| | |
|---|---|
| | WEIGHT |
| MATERIALS | PERCENT |

sequence 1

| | |
|---|---|
| acrylates copolymer (Covacryl A15) | 7.68 |
| acrylates copolymer (Covacryl E14) | 3.42 |
| purified water | 35.90 |

sequence 2

| | |
|---|---|
| purified water | 10.00 |
| titanium dioxide | 1.42 |
| iron oxides (pure oxy red 78054/3080) | 0.63 |
| D&C red no. 7 calcium lake (C19-011) | 0.60 |
| iron oxides (cosmetic yellow C-33-8073/CG490) | 0.34 |
| iron oxides (pure oxy black 7053/3068) | 0.25 |

sequence 3

| | |
|---|---|
| mica/titanium dioxide (Timiron MP-1005 supersilk) | 1.00 |

sequence 4

| | |
|---|---|
| PEG-90 diisostearate (Hydramol PGDS) | 3.00 |
| PEG/PPG-8/3 laurate (Hydramol PGPL) | 3.00 |

sequence 5

| | |
|---|---|
| purified water | 30.00 |
| hectorite (Bentone EW) | 0.60 |
| caprylyl glycol/phenoxyethanol/hexylene glycol | 0.50 |
| ethylhexylglycerin | 0.50 |

sequence 6

| | |
|---|---|
| purified water | 1.16 |
| TOTAL | 100.00 |

With reference to Table 6, sequence 1 materials are mixed until uniform. Sequence 2 materials are milled together until uniform and added to the sequence 1 mixture. Sequence 3 and 4 materials are sequentially added to the above mixture, mixing until uniform. Sequence 5 materials are premixed, heated to 75° C. until uniform, and added to the above mixture. The mixture is then mixed at room temperature until uniform. The product prepared is glossy, vivid in color, transfer-resistant, and comfortable on the lips.

### Example 7

### Lip Gloss Formulation

TABLE 7

| | |
|---|---|
| | WEIGHT |
| MATERIALS | PERCENT |

sequence 1

| | |
|---|---|
| acrylates copolymer (Covacryl A15) | 7.68 |
| acrylates copolymer (Covacryl E14) | 3.42 |
| purified water | 35.90 |

sequence 2

| | |
|---|---|
| PEG-90 diisostearate (Hydramol PGDS) | 3.00 |
| PEG/PPG-8/3 laurate (Hydramol PGPL) | 3.00 |

sequence 3

| | |
|---|---|
| purified water | 30.00 |
| caprylyl glycol/phenoxyethanol/hexylene glycol | 0.50 |
| ethylhexylglycerin | 0.50 |

sequence 4

| | |
|---|---|
| purified water | 16.00 |
| TOTAL | 100.00 |

The composition of Example 7 is prepared in the same manner as the composition of Example 6 except that the latter is an unpigmented lipgloss.

### Example 8

### Comparative Gloss Test

TABLE 8

| | |
|---|---|
| | WEIGHT |
| MATERIALS | PERCENT |

sequence 1

| | |
|---|---|
| acrylates copolymer (Covacryl | 9.80 |
| A15) | |
| acrylates copolymer (Covacryl | 4.00 |
| E14) | |
| caprlyl glycol/phenoxyethanol/hexylene glycol | 0.15 |
| purified water | 32.10 |

sequence 2

| | |
|---|---|
| purified water | 23.94 |
| caprlyl glycol/phenoxyethanol/hexylene glycol | 0.15 |
| Ethylhexylglycerin | 0.13 |
| iron oxides (cosmetic black) | 12.75 |

sequence 3

| | |
|---|---|
| kaolin | 6.30 |
| caprlyl glycol/phenoxyethanol/hexylene glycol | 0.20 |

sequence 4

| | |
|---|---|
| PEG-90 diisostearate (Hydramol PGDS) | 2.70 |
| PEG/PPS-8/3 laurate (Hydramol PGPL) | 1.28 |
| purified water | 6.30 |
| caprlyl glycol/phenoxyethanol/hexylene glycol | 0.20 |
| TOTAL | 100.00 |

An eyeliner formulation having the components shown in Table 8 is prepared by mixing sequence 1 materials together at room temperature until uniform, milling together sequence 2 materials, adding the milled sequence 2 materials to the sequence 1 mixture and mixing until uniform. Sequence 3 materials are added to the mixture of sequence 1 and 2 materials, and followed by the addition of the sequence 4 materials and mixing until uniform. Test samples (5 mm wet thickness) of the formulation of the invention (sample A) and three comparative eyeliner product emulsion formulations (samples B, C and D) are cast on Leneth card (form 2A-Opacity) within a 1″×2″ area. The samples are sufficiently thick (opaque) to avoid background reflectance. The samples are permitted to dry. Light is flashed at the samples at angles of 20° and 60°. The reflectance (scale of 0-100 gloss units) for each sample is measured at angles of 20° and 60°, using a gloss meter from BYK Garden; Model: micro-TRI-gloss dried. As indicated in Table 9 below, the inventive composition (A) demonstrated a higher gloss than any of the comparative emulsion compositions (B-D).

TABLE 9

GLOSS
UNITS

| SAMPLE | 20° | 60° |
|---|---|---|
| A (inv) | 0.5 | 5.4 |
| B (comp) | 0.2 | 2.1 |
| C (comp) | 0.1 | 0.4 |
| D (comp) | 0.1 | 1.7 |

Note:
Comparative sample B contains greater than 0.2 weight percent oil (Carnauba wax).
Comparative sample C contains greater than 0.2 weight percent oil (neopentyl glycol/C13-C14 isoparaffin).
Comparative sample C contains greater than 0.2 weight percent oil (oleyl alcohol).

### Example 9

### Evaluation of Water-Based Eyeliner for Transfer-Resistance

A study is done to determine the resistance of the eyeliner formulation shown in Table 8 to wear and flaking. Eight adult women participate in the study. The volunteer panelists are in normal health with no evidence of a systemic illness, nor any dermatogical disorder in the areas used in the study, which conditions might interfere with the analysis of the test results. Pregnant or lactating volunteers are excluded from the study. Panelists used for the study are not using systemic or topical retinoids, antihistamines or similar agents during the course of the study and two weeks prior to commencement of the study.

The women are instructed to wear no moisturizer or makeup for the test. The test sites are the eyelids. The panelists are given a sample of liquid eyeliner and instructed to apply the eyeliner as evenly as possible to the top and the bottom eyelids.

Wear and flaking evaluations are carried out immediately after product application, and two, four, six and eight hours after application.

Wear and flaking are assessed via photography using a Fuji S2 digital camera. The panelist's head is fixed in a headrest (Canfield Scientific) to which the camera is mounted. The camera lens is set at a distance of 0.35 m from the area to be photographed. Close up photographs of the eye area are taken at two, four, six and eight hours after application. The photographs are stored and viewed on Canfield Photofile Image Management Software (version 4.5.148). Clinical evaluations of the photographs are conducted by a trained investigator using a 10-point analog scale (shown below). The investigator is trained and qualified by an outside consultant to objectively identify and quantify the characteristics of skin parameters. The investigator has an extensive perceptual vocabulary, and is experienced in scale usage and the use of standardized evaluation techniques. A standard lexicon and references specifically for each of wear and flaking parameters (i.e. a photo scale depicting what a “0” looks lie, what a “2’ looks like, etc. up to “10”) are used for evaluation. “Wear” is defined as the visual observation of the amount of product color appearing and the amount of natural skin color that is visible at the indicated time points after application. “Flaking” is defined as the visual observation of the number of pieces of product falling onto the skin around the eyes or into the eyes at the indicated time points after application.

10 point scale

| | |
|---|---|
| 0 | 10 |
| No wear | Extreme wear |
| No flaking | Extreme flaking |

the results of the clinical evaluation are shown in Table 10 below. After 8 hours of wear, the eyeliner composition of the invention demonstrated minimal wear and minimal flaking.

TABLE 10

| Time After Application | Observed Wear | | Observed Flaking | |
|---|---|---|---|---|
| 0 | 0.0 | (none) | 0.0 | (none) |
| 2 | 0.0 | (none) | 0.0 | (none) |
| 4 | 0.0 | (none) | 0.0 | (none) |
| 6 | 0.4 | (minimal) | 0.0 | (none) |
| 8 | 0.8 | (minimal) | 0.7 | (minimal) |

While the invention has been described in connection with preferred embodiments, it not intended to limit the scope of the invention to the particular forms set forth, but it is intended to cover such alternative modifications and equivalents as may be included within the spirit and scope of the invention as defined by the appended claims.

We claim:

1. A transfer-resistant, single phase aqueous cosmetic composition, comprising one or more of each of a water-soluble film-forming acrylic acid or methacrylic acid-containing acrylates copolymer, a water-soluble ester and a pigment.

2. The cosmetic composition according to claim 1, wherein the acrylates copolymer comprises acrylic acid or methacrylic acid and a comonomer selected from the group consisting of alkyl and alkoxyl acrylates and alkyl and alkoxyl methacrylates.

3. The cosmetic composition according to claim 2, wherein the comonomer comprises a C1-C8 alkyl acrylate or methacrylate or a C1-C4 alkoxy acrylate or methacrylate.

4. The cosmetic composition according to claim 3, wherein the comonomer is selected from the group consisting of methyl acrylate, methyl methacrylate, ethyl acrylate, ethyl methacrylate, propyl acrylate, propyl methacrylate, isopropyl acrylate, isopropyl methacrylate, tertiary butyl acrylate, tertiary butyl methacrylate, isobutyl acrylate, isobutyl methacrylate, pentyl acrylate, pentyl methacrylate, isopentyl acrylate, isopentyl methacrylate, neopentyl acrylate, neopentyl methacrylate, hexyl acrylate, hexyl methacrylate, isohexyl acrylate, isohexyl methacrylate, heptyl acrylate, heptyl methacrylate, isoheptyl acrylate, isoheptyl methacrylate, octyl acrylate, octyl methacrylate, isooctyl acrylate, isooctyl methacrylate, 2-ethylhexyl acrylate, and 2-ethylhexyl methacrylate.

5. The composition according to claim 4, wherein the copolymer is selected from the group consisting of ethyl acrylate/methyl methacrylate/methacrylic acid; ethyl acrylate/methyl methacrylate/acrylic acid; ethyl acrylate/ethyl methacrylate/methacrylic acid; ethyl acrylate/ethyl methacrylate/acrylic acid; methyl acrylate/methyl methacrylate/methacrylic acid; methyl acrylate/methyl methacrylate/acrylic acid; methyl acrylate/ethyl methacrylate/methacrylic acid; and methyl acrylate/ethyl methacrylate/acrylic acid.

6. The composition according to claim 5, wherein the copolymer is ethyl acrylate/methyl methacrylate/methacrylic acid or ethyl acrylate/methyl methacrylate/acrylic acid.

7. The cosmetic composition according to claim 1, wherein the water-soluble acrylates copolymer is present in an amount of from about 1 to about 95 percent by weight of the total composition.

8. The cosmetic composition according to claim 7, wherein the water-soluble acrylates copolymer is present in an amount of from about 5 to about 70 percent by weight of the total composition.

9. The cosmetic composition according to claim 8, wherein the water-soluble acrylates copolymer is present in an amount of from about 10 to about 30 percent by weight of the total composition.

10. The cosmetic composition according to claim 1, wherein the water-soluble ester is selected from the group consisting of polyether derivatives, polyoxypropylene derivatives, glycol derivatives and glycerin derivatives.

11. The cosmetic composition according to claim 11, wherein the water-soluble ester is polyglyceryl-3 laurate, PEG-90 diisostearate, PEG/PPG-8/3 laurate, PEG/PPG-8/3 diisostearate, triisostearoyl polyglyceryl-3 dimer dilinoleate, or a mixture of any two or more of said esters.

12. The cosmetic composition according to claim 1, wherein the water-soluble ester is present in an amount of from about 1 to about 60 percent by weight of the total composition.

13. The cosmetic composition according to claim 12, wherein the water-soluble ester is present in an amount of from about 2 to about 20 percent by weight of the total composition.

14. The cosmetic composition according to claim 1, wherein the ratio of the water-soluble acrylates copolymer to the water-soluble ester is greater than 1:1.

15. The cosmetic composition according to claim 14, wherein the ratio of the water-soluble acrylates copolymer to the water-soluble ester is 2:1 to 20:1.

16. The cosmetic composition according to claim 15, wherein the ratio of the water-soluble acrylates copolymer to the water-soluble ester is 2:1 to 4:1.

17. The cosmetic composition according to claim 1, wherein the pigment is selected from the group consisting of cosmetically acceptable inorganic and organic pigments.

18. The cosmetic composition according to claim 17, wherein the inorganic pigment comprises a red, blue, black, green or yellow iron oxide, titanium dioxide, bismuth oxychloride or a mixture of any two or more of said inorganic pigments.

19. The cosmetic composition according to claim 17, wherein the organic pigment is selected from the group consisting of red, orange, yellow, green, blue, violet, and lakes thereof.

20. The cosmetic composition according to claim 18, wherein the pigment is a black or a red iron oxide.

21. The cosmetic composition according to claim 1, wherein the pigment is present in an amount of from about 1 to about 20 percent by weight of the total composition.

22. The cosmetic composition according to claim 21, wherein the pigment is present in an amount of from about 5 to about 15 percent by weight of the total composition.

23. The cosmetic composition according to claim 1, which is an eyeliner, a mascara, a concealer, a lipgloss or a lipliner.

24. The cosmetic composition according to claim 1, in which oil, if present, is present in an amount of no greater than 0.2 weight percent.

25. A transfer-resistant, single phase aqueous cosmetic composition comprising from about 10 to about 30 weight percent ethyl acrylate/methylmethacrylate/methacrylic acid copolymer, from about 2 to about 20 weight percent PEG-90 diisostearate and PEG/PPG-8/3-laurate, and from about 5 to about 15 weight percent black iron oxide, based on the total weight of the composition.

26. A transfer-resistant, single phase aqueous cosmetic composition comprising from about 10 to about 30 weight percent ethyl acrylate/methylmethacrylate/methacrylic acid copolymer, from about 2 to about 20 weight percent PEG-90 diisostearate, and from about 5 to about 15 weight percent black iron oxide, based on the total weight of the composition.

27. A transfer-resistant, single phase aqueous cosmetic composition comprising one or more of each of a film-forming water-soluble acrylic acid or methacrylic acid-containing acrylates copolymer, and a water-soluble ester.

28. The cosmetic composition according to claim 27, wherein the acrylates copolymer comprises acrylic acid or methacrylic acid and a comonomer selected from the group consisting of alkyl and alkoxyl acrylates and alkyl and alkoxyl methacrylates.

29. The cosmetic composition according to claim 28, wherein the comonomer comprises a C1-C8 alkyl acrylate or methacrylate or a C1-C4 alkoxy acrylate or methacrylate.

30. The cosmetic composition according to claim 29, wherein the comonomer is selected from the group consisting methyl acrylate, methyl methacrylate, ethyl acrylate, ethyl methacrylate, propyl acrylate, propyl methacrylate, isopropyl acrylate, isopropyl methacrylate, tertiary butyl acrylate, tertiary butyl methacrylate, isobutyl acrylate, isobutyl methacrylate, pentyl acrylate, pentyl methacrylate, isopentyl acrylate, isopentyl methacrylate, neopentyl acrylate, neopentyl methacrylate, hexyl acrylate, hexyl methacrylate, isohexyl acrylate, isohexyl methacrylate, heptyl acrylate, heptyl methacrylate, isoheptyl acrylate, isoheptyl methacrylate, octyl acrylate, octyl methacrylate, isooctyl acrylate, isooctyl methacrylate, 2-ethylhexyl acrylate, and 2-ethylhexyl methacrylate.

31. The composition according to claim 30, wherein the copolymer is selected from the group consisting of ethyl acrylate/methyl methacrylate/methacrylic acid; ethyl acrylate/methyl methacrylate/acrylic acid; ethyl acrylate/ethyl methacrylate/methacrylic acid; ethyl acrylate/ethyl methacrylate/acrylic acid; methyl acrylate/methyl methacrylate/methacrylic acid; methyl acrylate/methyl methacrylate/acrylic acid; methyl acrylate/ethyl methacrylate/methacrylic acid; and methyl acrylate/ethyl methacrylate/acrylic acid.

32. The composition according to claim 31, wherein the copolymer is ethyl acrylate/methyl methacrylate/methacrylic acid or ethyl acrylate/methyl methacrylate/acrylic acid.

33. The cosmetic composition according to claim 27, wherein the water-soluble acrylate copolymer is present in an amount of from about 1 to about 95 percent by weight of the total composition.

34. The cosmetic composition according to claim 33, wherein the water-soluble acrylate copolymer is present in an amount of from about 5 to about 70 percent by weight of the total composition.

35. The cosmetic composition according to claim 34, wherein the water-soluble acrylate copolymer is present in an amount of from about 10 to about 30 percent by weight of the total composition.

36. The cosmetic composition according to claim 27, wherein the water-soluble ester is selected from the group consisting of polyether derivatives, polyoxypropylene derivatives, glycol derivatives and glycerin derivatives.

37. The cosmetic composition according to claim 36, wherein the water-soluble ester is polyglyceryl-3 laurate, PEG-90 diisostearate, PEG/PPG-8/3 laurate, PEG/PPG-8/3 diisostearate, triisostearoyl polyglyeryl-3 dimer dilinoleate, or a mixture of any two or more of said esters.

38. The cosmetic composition according to claim 27, wherein the water-soluble ester is present in an amount of from about 1 to about 60 percent by weight of the total composition.

39. The cosmetic composition according to claim 38, wherein the water-soluble ester is present in an amount of from about 2 to about 20 percent by weight of the total composition.

40. The cosmetic composition according to claim 27, wherein the ratio of the water-soluble acrylate copolymer to the water-soluble ester is greater than 1:1.

41. The cosmetic composition according to claim 40, wherein the ratio of the water-soluble acrylates copolymer to the water-soluble ester is 2:1 to 20:1.

42. The cosmetic composition according to claim 41, wherein the ratio of the water-soluble acrylates copolymer to the water-soluble ester is 2:1 to 4:1.

43. The cosmetic composition according to claim 27, in which oil, if present, is present in an amount of no greater than 0.2 weight percent.

44. A transfer-resistant, single phase aqueous cosmetic composition comprising from about 10 to about 30 weight percent ethyl acrylate/methylmethacrylate/methacrylic acid copolymer, and from about 2 to about 20 weight percent PEG-90 diisostearate and PEG/PPG-8/3-laurate.

45. A transfer-resistant, single phase aqueous cosmetic composition comprising from about 10 to about 30 weight percent ethyl acrylate/methylmethacrylate/methacrylic acid copolymer, and from about 2 to about 20 weight percent PEG-90 diisostearate.

46. A method of improving the transfer-resistance and/or the shine of a cosmetic composition, comprising combining:
water;
one or more water-soluble film-forming acrylic acid- or methacrylic acid-containing acrylates copolymers; and
one or more water-soluble esters;
wherein the composition contains substantially no oil.

47. A method of improving the transfer-resistance and/or the shine and/or the color intensity of a cosmetic composition, comprising combining:
water;
one or more water-soluble film-forming acrylic acid- or methacrylic acid-containing acrylates copolymers;
one or more water-soluble esters; and
pigment;
wherein the composition contains substantially no oil.

48. A method of redefining the lipline, comprising:
providing a transfer-resistant single phase aqueous composition comprising a water-soluble film-forming acrylic acid- or methacrylic acid-containing acrylates copolymer, a water-soluble ester, and a pigment having a natural lip color, and a transfer-resistant single phase aqueous composition comprising a water-soluble film-forming acrylic acid- or methacrylic acid-containing acrylates copolymer, a water-soluble ester, and a pigment having a desired color;
dipping a lipliner brush into the composition having the natural lip color so as to load the brush with the composition;
tracing a line with the lipliner brush just inside or just outside a user's natural lipline;
allowing the traced line to dry;
dipping a lipliner brush into the composition having the desired lip color so as to load the brush with the composition; and applying the desired color composition within the redefined lipline.

49. A method according to claim 48, which further comprises applying over the desired shade a lipgloss composition comprising a transfer-resistant single phase aqueous composition comprising a water-soluble film-forming acrylic acid- or methacrylic acid-containing acrylates copolymer and a water-soluble ester.

50. A method according to claim 49, wherein the lipgloss composition further comprises a pigment.
